(19) United States
(12) Patent Application Publication
(10) Pub. No.: US 2021/0154415 A1
(43) Pub. Date: May 27, 2021

(54) Electronic Smart Syringe

(71) Applicant: Vipin Vangara, Charlotte, NC (US)

(72) Inventors: Ravi Chandar Vangara, Bangalore (IN); Rajesh Vangara, Concord, NC (US); Siddharth Vangara, Concord, NC (US)

(21) Appl. No.: 16/555,911

(22) Filed: Aug. 29, 2019

(51) Int. Cl. A61M 5/315; A61K 9/00; A61K 38/28; G06K 9/00

(52) U.S. Cl. CPC A61M 5/31566; A61M 5/31568; A61M 5/31548; A61K 9/0019; A61M 2005/3125; A61K 9/0009; G06K 9/00013; A61M 2202/048; A61M 2205/3584; A61K 38/28

(57) ABSTRACT

An electronic smart syringe is disclosed. The electronic smart syringe includes a barrel. The barrel includes a slot on a proximal end. The electronic smart syringe also includes a needle coupled on a distal end of the barrel. The electronic smart syringe further includes a plunger coupled on the proximal end of the barrel via the slot. The electronic smart syringe further includes a cartridge secured within the barrel and coupled with the plunger. The cartridge is configured to expel, via the needle, a prescribed dosage of a drug in the body of a patient. The electronic smart syringe further includes a biometric access panel operatively coupled with the plunger. The biometric access panel is configured to authenticate an authorized user for expelling the drug upon receiving one or more fingerprints. Electronic Smart syringe includes a flexible display unit and synchronized to the external end user computing device.

### BACKGROUND

Embodiments of a present disclosure relate to a drug injection device and more particularly to an electronic smart syringe.

Many drugs are intended to be administered parenterally, because they are not resorbed sufficiently. A typical example includes peptide drugs like insulin. The pharmacological half-life of such drugs is very short which leaves the need for multiple daily injections. An insulin-dependent diabetic patient needs to inject multiple times a day at variable doses, adjusted to food intake and physical activity. Therefore, often the injections are performed by the patient himself instead of trained medical professionals. However, old age people may not be able to adjust the dosage of the insulin or end up wrongly adjusting the insulin dosage. Hence, to comfort the patients while dealing with frequent variable dose, variety of self-administrations drug delivery devices have been introduced. Also there are 0.5 to 1% patient deaths are due to wrong anaesthesia administered, and its not clear who administered the dosage and how much dosage was recommended based on age, weight or the like, with the currently available syringes in the market.

One such kind of self-administration drug delivery device is called “insulin pen”. Such pens are critical medical devices for the reason that both an overdose and under dose may lead to severe health problems including death. Also, such injection pen facilitates the multiple injections required. However, using such pens it is difficult and cumbersome for the patient to keep records of all parameters determining the insulin dose adjustment and dosage administered record. Also, such insulin pen does not notify to the condition of the insulin pen like the quantity of insulin or likes left, usage of the needle, last dosage, next dosage due, over dosage, under dosage, missed dosage and the like. Similarly, the insulin pen does not alert over missed or excess dosage to the patient or to their kith and kin and does not have an interface with the smart phone to send notifications to Kith and Kin, healthcare providers and to self. electronic smart syringe

Furthermore, currently available self-administration drug delivery device has security issues, as any unauthorised person may use the insulin pen to harm the patient.

Hence, there is a need for an improved electronic smart syringe to address the aforementioned issue(s).

### BRIEF DESCRIPTION

In accordance with an embodiment of the present disclosure, an electronic smart syringe is provided. The electronic smart syringe includes a barrel. The barrel includes a slot on a proximal end. The electronic smart syringe also includes a needle coupled on a distal end of the barrel. The electronic smart syringe further includes a plunger coupled on the proximal end of the barrel via the slot. The electronic smart syringe further includes a cartridge secured within the barrel and coupled with the plunger. The cartridge is configured to expel, via the needle, a prescribed dosage of a drug in the body of a patient. The electronic smart syringe further includes a biometric access panel operatively coupled with the plunger. The biometric access panel is configured to authenticate an authorized user for expelling the drug upon receiving one or more fingerprints

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF THE DRAWINGS

The disclosure will be described and explained with additional specificity and detail with the accompanying figures in which:

FIG. 1 is a schematic representation of an electronic smart syringe in accordance with an embodiment of the present disclosure;

FIG. 2 is a schematic representation of the electronic smart syringe with a biometric panel in accordance with an embodiment of the present disclosure; and

FIG. 3 is a schematic representation of one embodiment of the electronic smart syringe of FIG. 1 in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more devices or sub-systems or elements or structures or components preceded by “comprises . . . a” does not, without more constraints, preclude the existence of other devices, sub-systems, elements, structures, components, additional devices, additional sub-systems, additional elements, additional structures or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

Embodiments of the present disclosure relate to an electronic smart syringe. The electronic smart syringe includes a barrel. The barrel includes a slot on a proximal end. The electronic smart syringe also includes a needle coupled on a distal end of the barrel. The electronic smart syringe further includes a plunger coupled on the proximal end of the barrel via the slot. The electronic smart syringe further includes a cartridge secured within the barrel and coupled with the plunger. The cartridge is configured to expel, via the needle, a prescribed dosage of a drug in the body of a patient. The electronic smart syringe further includes a biometric access panel operatively coupled with the plunger. The biometric access panel is configured to authenticate an authorized user for expelling the drug upon receiving one or more fingerprints.

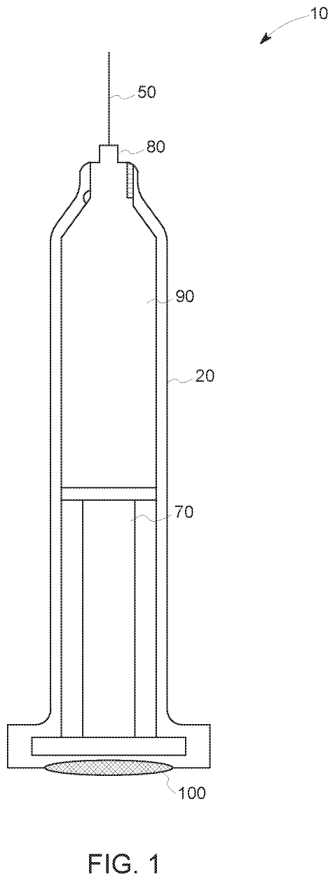

FIG. 1 is a schematic representation of an electronic smart syringe 10 in accordance with an embodiment of the present disclosure. The electronic smart syringe 10 includes a barrel 20. The barrel 20 includes a slot (not shown in FIG. 1) on a proximal end of the barrel 20. The electronic smart syringe 10 also includes a needle 50 which is coupled to a distal end of the barrel 20. In one embodiment, the needle 50 may be a hypodermic needle which may be used for intradermal, intramuscular and subcutaneous injection. As used herein, the intradermal, the intramuscular and the subcutaneous injection targets a different skin layer such as subcutaneous injections are administered in the fat layer, underneath the skin whereas the intramuscular injections are delivered into the muscle.

Furthermore, the electronic smart syringe 10 includes a plunger 70 which is coupled to the proximal end of the barrel 20 via the slot. The plunger 70 may be linearly pulled and pushed along the inside of the barrel 20, allowing the needle 50 to take in and expel drug through a discharge orifice 80 at a front end of the barrel 20. The front end of the barrel 20 is fitted with the needle 50 to flow out the drug. In one embodiment, the drug may be insulin, anaesthesia, asthma related drug or the like.

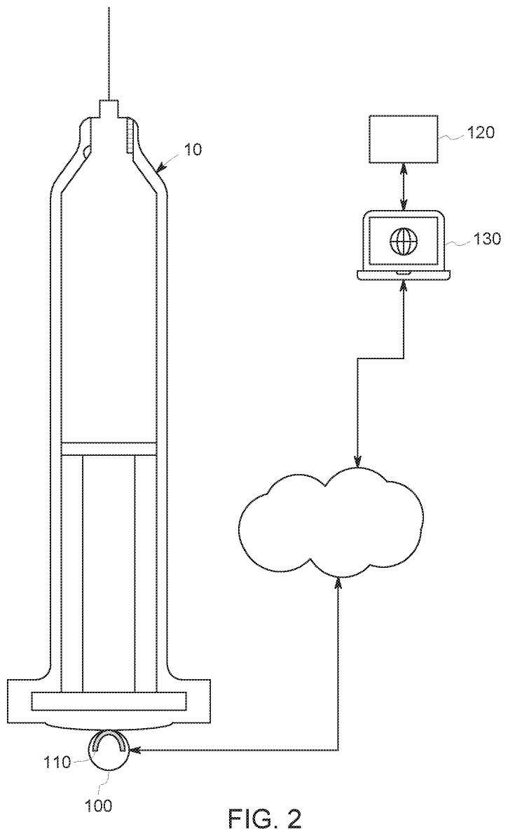

Moreover, the electronic smart syringe 10 further includes a cartridge 90 which is secured within the barrel 20. In one embodiment, the barrel 20 may include a recess which is adapted to receive the cartridge 90. The cartridge 90 is also coupled to the plunger 70. The cartridge 90 is configured to expel, via the needle 50, a prescribed dosage of the drug in the body of a patient. In another embodiment, the cartridge 90 may be a rigid cartridge. In some embodiments, the cartridge 90 may be disposable cartridge. The electronic smart syringe 10 further includes a biometric access panel 100 operatively coupled to the plunger 70. The biometric access panel 100 is configured to authenticate an authorized user for expelling the drug upon receiving one or more fingerprints. One embodiment of the biometric access panel 100 is described in FIG. 2.

FIG. 2 is a schematic representation of the electronic smart syringe 10 with the biometric access panel 100 in accordance with an embodiment of the present disclosure. The biometric access panel 100 includes a fingerprint sensor 110. The fingerprint sensor 110 is configured to receive one or more fingerprints (digital image of the fingerprint patterns) from one or more authorized users. In one embodiment, the one or more authorized users may be the patient, family members of the patient or a healthcare provider such as nurse doctor or the like. Upon receiving the fingerprints from the one or more authorized users, the fingerprints of the one or more authorized user are being saved in a memory 120 of an external computing device 130 as reference fingerprints. When a user enters his or her fingerprints using the fingerprint sensor, these fingerprints are being send to the external computing device 130. Upon receiving the fingerprints, these fingerprints are compared with the reference fingerprints to authenticate the user as the authorized user, where the authorized user may access the electronic smart syringe 10 for expelling the drug in the body of the patient. Fingerprint registering may be carried with the help of smart phone.

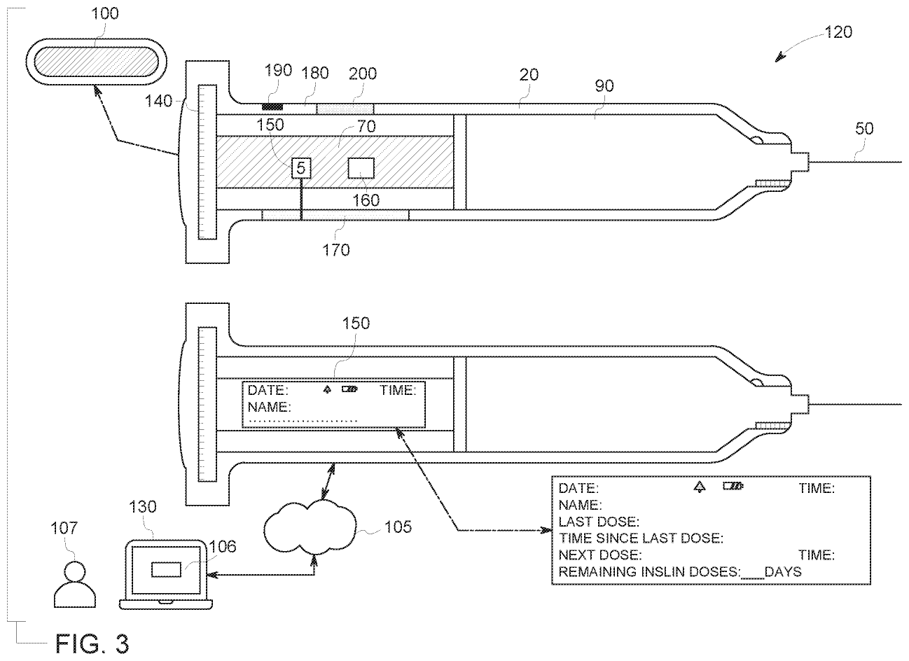

FIG. 3 is a schematic representation of one embodiment of the electronic smart syringe 10 of FIG. 1 in accordance with an embodiment of the present disclosure. The electronic smart syringe 10 includes a barrel 20. The barrel 20 includes a slot (not shown in FIG. 3) on a proximal end of the barrel 20. The electronic smart syringe 10 also includes a needle 50 which is coupled to a distal end of the barrel 20. Further, the electronic smart syringe 10 includes a plunger 70 which is coupled to the proximal end of the barrel 20 via the slot. The electronic smart syringe 10 further includes a cartridge 90 which is secured within the barrel 20. The cartridge 90 is configured to expel, via the needle 50, a prescribed dosage of the drug in the body of a patient. The electronic smart syringe 10 includes a biometric access panel 100 operatively coupled to plunger 70. The biometric access panel 100 is configured to authenticate an authorized user 107 for expelling the drug upon receiving one or more fingerprints.

Considering an example where the electronic smart syringe 10 is communicatively coupled to an external computing device 130 such as a smart phone via a smart phone interface 106. The smart phone interface 106 is configured to enable the registration of the authorized user 107. The authorized user 107 may be a patient and a healthcare provider. In some embodiments, the external computing device 130 may include computer, laptop, personal digital assistant (dedicated or generic), cellular smart phone or the like. The electronic smart syringe 10 and the smart phone interface 106 may communicate with each other via a communication network 105. In one embodiment, the communication network 105 may include a wired communication medium such as local area network (LAN). In another embodiment, the communication network 105 may include a wireless communication medium such as radio frequency (RF) communication, Bluetooth, Infrared (IR) communication, wireless fidelity (wi-fi) or the like.

In one embodiment, the electronic smart syringe 10 may include a dosage administration unit 140 operatively coupled to a first surface of the barrel 20. The dosage administration unit 140 is configured to enable the authorized user to administer dosage of the drug. In such embodiment, the dosage administration unit 140 may enable the authorized user to administer the prescribed dosage of the drug remotely by the external computing device 130 through the communication network.

Continuing the same example where a healthcare provider may administer the electronic smart syringe 10 via dosage administration unit 140 using the external computing device 130 such as smart phone through wi-fi network. The healthcare provider may send a signal through his smart phone via the smart phone interface 106 and once the electronic smart syringe is in synchronization, the dosage administration unit 140 enable the authorized user to set the prescribed dosage of the drug. In another embodiment, the dosage administration unit 140 is configured to enable the authorized user to administer the prescribed dosage of the drug by a dosage calibration dial. The dosage calibration dial is operatively coupled with the barrel 20 of the electronic smart syringe 10. The authorized user may operate the onboard dosage calibration dial to set the dosage to be taken by the patient. In such embodiment, the calibration dial may be a mechanical dial, a digital dial or the like.

In one embodiment, the dosage administration unit 140 may enable the authorized user to lock the expelling mechanism for the prescribed dosage of the drug. The dosage administration unit 140 remotely (via smart phone interface) receives a command from the authorized user. Based on the received command, the electronic smart syringe 10 may get locked with the prescribed dosage of the drug to be expel in the body of the patient. In one embodiment, the electronic smart syringe 10 also includes a Flexible display panel 150 which is operatively coupled to the barrel 20. The display panel 150 is configured to display at least one of dosage status, cartridge status, needle status, battery status or the combination thereof. In one embodiment, the display panel 150 may be a liquid crystal display panel. In another embodiment, the display panel 150 may be a flexible display.

In some embodiments, the dosage status may include one or more missed drug dosages, elapsed drug dosage, upcoming drug dosage and drug dosage deviated from the prescribed dosage (excess or lower dosage of drug than the prescribed dosage). In one embodiment, the cartridge status may include quantity of drug remaining in the cartridge, refill status of the cartridge and replacement status of the cartridge (replacement of a used cartridge with an unused cartridge). In a specific embodiment, the needle status may include status of last needle changed, a plurality of barrels carried with the needle and a usage status of the needle. In a preferred embodiment, an expiry date status of the Insulin cartridge may also be notified to the smart phone, subject to registering the cartridge life in the smart phone interface.

In one embodiment, the electronic smart syringe 10 may further include a communication unit 160 operatively coupled to the barrel 20. The communication unit 160 is configured to communicate one or more alerts to the external computing device 130 of the authorized user. The one or more alerts may include at least one dosage status, cartridge status, needle status and an alert to calibrate dosage. In such embodiment, the one or more alerts may be in at least one of the form such as an audio alert or a visual alert or the combination thereof.

In some embodiments, the electronic smart syringe 10 may include a control unit 170 which is configured to control the electronic smart syringe 10 remotely. In one embodiment, the electronic smart syringe 10 may further include a charging unit 180. The charging unit 180 may include a charging slot 190 secured within at least one side of the barrel 20. The charging unit 180 may also include a battery 200. In one embodiment, the battery 200 may be a rechargeable battery. In another embodiment, the battery 200 may be a replaceable/disposable battery. The charging unit 180 is configured to charge the battery 200 by receiving electrical charges from one or more external energy sources 130 via the charging slot 190.

Various embodiments of the electronic smart syringe described above enables a biometric authentication of the patient by a fingerprint sensor to exclude confounding or tampering with data belonging to patient. The electronic smart syringe provides a secure and reliable mechanism to prevent misuse of the electronic and excess/high insulin/anaesthesia administration than the prescribed dosages. electronic smart syringe. The electronic syringe is equipped with a flexible display to various vital information pertaining to dosage, next dosage due, previous dosage, excess, low or missed dosage on specific times and have good interface with the smart phone for alerts at regular intervals.

Furthermore, the display panel and communication unit are mounted directly onto the syringe barrel, thus providing a totally self-contained syringe system that is completely and which provides increased convenience in terms of maintaining sterility and portability.

It will be understood by those skilled in the art that the foregoing general description and the following detailed description are exemplary and explanatory of the disclosure and are not intended to be restrictive thereof.

While specific language has been used to describe the disclosure, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, order of processes described herein may be changed and are not limited to the manner described herein. Moreover, the actions of any flow diagram need not be implemented in the order shown; nor do all of the acts need to be necessarily performed. Also, those acts that are not dependent on other acts may be performed in parallel with the other acts. The scope of embodiments is by no means limited by these specific examples.

We claim:

1. An electronic smart syringe comprising:
a barrel; wherein the barrel comprises a slot on a proximal end;
a needle coupled on a distal end of the barrel;
a plunger coupled on the proximal end of the barrel via the slot;
a cartridge secured within the barrel and coupled with the plunger; wherein the cartridge is configured to expel, via the needle, a prescribed dosage of a drug in the body of a patient; and
a biometric access panel operatively coupled to plunger, wherein the biometric access panel is configured to authenticate an authorized user for expelling the drug upon receiving one or more fingerprints.

2. The electronic smart syringe of claim 1, wherein the needle comprises a hypodermic needle.

3. The electronic smart syringe of claim 1, wherein the barrel comprises a recess adapted to receive the cartridge.

4. The electronic smart syringe of claim 1, wherein the drug comprises insulin, anaesthesia or asthma related drug.

5. The electronic smart syringe of claim 1, wherein the biometric access panel comprises a fingerprint sensor.

6. The electronic smart syringe of claim 1, wherein the authorized user comprises at least one of the patient and one or more healthcare providers.

7. The electronic smart syringe of claim 1, further comprising a dosage administration unit operatively coupled to the barrel, wherein the dosage administration unit is configured to enable the authorized user to administer a prescribed dosage of the drug.

8. The electronic smart syringe of claim 7, wherein the dosage administration unit enable the authorized user to lock the expelling mechanism for the prescribed dosage of the drug.

9. The electronic smart syringe of claim 7, wherein the dosage administration unit is configured to enable the authorized user to administer the prescribed dosage of the drug by an external computing device through a communication network.

10. The electronic smart syringe of claim 7, wherein the dosage administration unit is configured to enable the authorized user to administer the prescribed dosage of the drug by a dosage calibration dial.

11. The electronic smart syringe of claim 1, further comprising a display panel operatively coupled to the barrel, wherein the display panel is configured to display at least one of dosage status, cartridge status and needle status and battery status. Electronic smart syringe

12. The electronic smart syringe of claim 11, wherein the dosage status comprises one or more missed drug dosages, elapsed drug dosage, upcoming drug dosage and drug dosage deviated from the prescribed dosage.

13. The electronic smart syringe of claim 11, wherein the cartridge status comprises quantity of drug remaining in the cartridge, refill status of the cartridge and replacement status of the cartridge.

14. The electronic smart syringe of claim 11, wherein the needle status comprises status of last needle changed, a plurality of barrels carried with the needle and a usage status of the needle.

15. The electronic smart syringe of claim 11, wherein the display panel comprises a flexible display panel.

16. The electronic smart syringe of claim 1, further comprising a communication unit operatively coupled to the barrel, wherein the communication unit is configured to communicate one or more alerts to the external computing device, wherein the one or more alerts comprises at least one of dosage status, cartridge status, needle status and an alert to calibrate dosage.

17. The electronic smart syringe of claim 16, wherein the one or more alerts comprises at least one of an audio alert and a visual alert.

18. The electronic smart syringe of claim 1, further comprising a charging unit, wherein the charging unit comprises a charging slot secured within the barrel and a battery, wherein the charging unit is configured to charge the battery by receiving electrical charges via the charging slot.
